(19) United States
(12) Patent Application Publication
(10) Pub. No.: US 2010/0111917 A1
(43) Pub. Date: May 6, 2010

(54) CELL-ADHESIVE POLYELECTROLYTE MATERIAL FOR USE AS MEMBRANE AND COATING

(76) Inventors: Jackie Y. Ying, Singapore (SG); Andrew C.A. Wan, Singapore (SG); Karl M. Schumacher, Singapore (SG)

Correspondence Address: KLARQUIST SPARKMAN, LLP, 121 SW SALMON STREET, SUITE 1600, PORTLAND, OR, 97204, US

(21) Appl. No.: 12/293,801

(22) PCT Filed: Oct. 17, 2006
(86) PCT No.: PCT/SG2006/000305

§ 371 Date: Sep. 22, 2009

Related U.S. Application Data
(60) Provisional application No. 60/783,864, filed on Mar. 21, 2006.

(51) Int. Cl. A61K 38/43; C12N 5/00; A61K 47/00; A61K 38/16; A61K 38/02; A61K 31/7052; A61P 43/00

(52) U.S. Cl. 424/94.1; 435/395; 514/772; 514/12; 514/2; 514/44 R

(57) ABSTRACT

A multilayer polyelectrolyte support material is provided that includes a polyelectrolyte layer and a polyelectrolyte-polyethylene glycol layer adjacent to the polyelectrolyte layer. The support material also includes a ligand conjugated to the polyelectrolyte-polyethylene glycol layer, allowing for attachment of a protein or a cell to the support material with controlled orientation.

### CROSS-REFERENCE TO RELATED APPLICATION

This application claims benefit and priority from U.S. provisional patent application No. 60/783,864, filed on Mar. 21, 2006, the contents of which are incorporated herein by reference.

### FIELD OF IRE INVENTION

The present invention relates generally to membranes and coatings, and particularly to cell-adhesive membranes and coatings useful as cell support matrices and scaffolding and for coating medical devices.

### BACKGROUND OF THE INVENTION

The trend in biomaterials technology has been shifting away from one where new materials are developed based on trial-and-error to one where materials are engineered based on careful design that incorporates specific biological functionalities. This is particularly evident in the efforts to tailor biocompatible surfaces for medical devices and tissue engineering.

In the early generations of such materials, many surfaces were found to support cell attachment. These observed cell-adhesive properties were largely due to non-specific charge interactions between the cells and the surfaces, which were often enriched by the adsorption of proteins from the cell culture media. Such non-specific protein interactions are, however, indiscriminate in terms of ligand type and orientation of ligand attachment at the surface of the material.

Membranes of various types and compositions have been used as the components in implants and medical devices, such as guided periodontal tissue regeneration in dental applications,^{1,2} kidney hemodialysis,³ and as an epithelial equivalent for the conjurictiva.⁴

Thus, there remains a need for a biocompatible material that allows for organised, directed attachment of cells or proteins on the material surface while reducing non-specific adhesion of cells or proteins.

In one aspect, the present invention provides a support material comprising: a polyelectrolyte layer comprising a first polyelectrolyte; a polyelectrolyte-polyethylene glycol layer adjacent to the polyelectrolyte layer, the polyelectrolyte-polyethylene glycol layer comprising a second polyelectrolyte being of opposite charge to the first polyelectrolyte, the second polyelectrolyte conjugated to polyethylene glycol by a first functional group on the second polyelectrolyte and a second functional group on the polyethylene glycol; and a ligand conjugated to the polyelectrolyte-polyethylene glycol layer by a third functional group on the ligand and a fourth functional group on the polyelectrolyte-polyethylene glycol layer, wherein neither of the third or fourth functional groups are a carboxyl group or an amino group.

In another aspect, the present invention provides a method of forming the support material as described herein, the method comprising: providing a first layer comprising a first polyelectrolyte; applying a polyelectrolyte-polyethylene glycol conjugate to the first layer to form a second layer adjacent to the first layer, where in the polyelectrolyte-polyethylene glycol conjugate is formed by reacting a first functional group on a second polyelectrolyte and a second functional group on a polyethylene glycol, the second polyelectrolyte being of opposite charge to the first polyelectrolyte; and conjugating a ligand to the second layer by reacting a third functional group on the ligand with a fourth functional group on the second layer; wherein neither of the third or fourth functional groups are a carboxyl group or an amino group.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram of an exemplary embodiment of the material having a polycationic layer, a polyanionic-polyethylene glycol (PEG) layer and a ligand conjugated to the polyanionic-PEG layer;

FIG. 2 is a schematic diagram of an exemplary material, showing the respective chemistries of each of the particular layers in this embodiment of the material: (a) polycationic layer: silica cross-linked chitosan membrane/coating, (b) polyanion-PEG layer: cysteine alginate-PEG conjugate, which forms a polyelectrolyte complex with the bottom layer, and (c) ligand: RGD-containing peptide immobilized to the polyanion-PEG layer by maleimidyl chemistry;

FIG. 3 is a graph showing membrane swelling ratio as a function of TEOS:chitosan volume ratio;

FIG. 4 is light micrographs of primary human cortical cells cultured on (a) chitosan-alginate-PEG-RGD, (b) chitosan-alginate-PEG, (c) chitosan, (d) chitosan-heparin-PEG-RGD, and (e) chitosan-heparin-PEG membranes;

FIG. 5 is fluorescent micrographs of primary human cortical cells cultured on (a) chitosan-alginate-PEG-RGD, (b) chitosan-alginate-PEG, (c) chitosan, (d) chitosan-heparin-PEG-RGD, and (e) chitosan-heparin-PEG membranes. The red-stained cells express AQP1, as indicated by the arrows. DAPI staining (blue) displays the nuclei of the attached cells;

FIG. 6 is light micrographs of HepG2 cells cultured on glass coverslips with (a,b) no coating, (c,d) chitosan-alginate-PEG coating, and (e,f) chitosan-alginate-PEG-RGD coating—(b), (d) and (f) are taken at higher magnification; and

FIG. 7 (a) is a photograph of the swellable cell-adhesive polyelectrolyte membrane; (b) and (c) are fluorescence micrographs of human mesenchymal stem cells seeded onto the swellable polyelectrolyte membrane modified with an RGD peptide and unmodified, respectively.

### DETAILED DESCRIPTION

Methods to immobilize ligands on non-fouling surfaces used as cell supports are important since such methods allow for study of the effect of individual factors on cell adhesion, proliferation and differentiation without the confounding influence of protein adsorption from the serum or the extracellular matrices (ECM) produced by the cells themselves.¹¹ Previously, two prominent approaches to achieve non-cell-adhesive surfaces involved the use of PEG in its various forms (branched and linear),¹² and more recently, polyelectrolyte complexes.⁷ Lin and co-workers have shown that the coating of a chitosan-heparin conjugate on polyacrylonitrile membranes improved their hemocompatibility, as characterized by reduced protein adsorption, platelet adhesion and thrombus formation.¹³

The present invention relates to a material that is useful as scaffolding for cell attachment, and which can be used as a membrane or to coat biomedical devices and implants. The material comprises alternating layers of polyelectrolytes, with each layer of polyelectrolyte having opposite charge to that of an adjacent layer. The outermost layer, intended to be used either directly or indirectly as a surface for cell or protein adhesion, comprises a polyelectrolyte conjugated to polyethylene glycol (PEG). The material comprises a ligand for cell or protein attachment, conjugated to the polyelectrolyte-PEG layer. The polyelectrolyte provides charge groups to interact with the adjacent polyelectrolyte layer of opposite charge, the PEG provides a non-fouling surface with low affinity for non-specific protein interactions, while the ligand provides a capture molecule for specific adhesion of cells or proteins or other molecules to the outer layer of the material. To reduce the possibility of non-specific cross-reaction with functional groups on proteins, the PEG-electrolyte layer is conjugated via non-carboxyl and non-amino groups on the polyelectrolyte and on the PEG moieties, for example via a sulfhydryl or hydroxyl group on the polyelectrolyte or PEG reacting with an appropriate reactive functional group on the other of the polyelectrolyte or PEG. Similarly, the ligand for cell attachment is also conjugated via non-carboxyl and non-amino groups to the polyelectrolyte-PEG layer.

The polyelectrolyte conjugated to the PEG may be either a polyanion or a polycation. However, in order to reduce non-specific attachment of cells or proteins to the surface of the material, it may be desired to conjugate a polyanion to the PEG, as cells tend to have a greater tendency to bind non-specifically to certain polycations, for example, poly-L-lysine. In this way, the material can be designed such that adhesion of cells or proteins to the surface of the material is predominantly via interactions with a ligand conjugated to the PEG-polyanion layer.

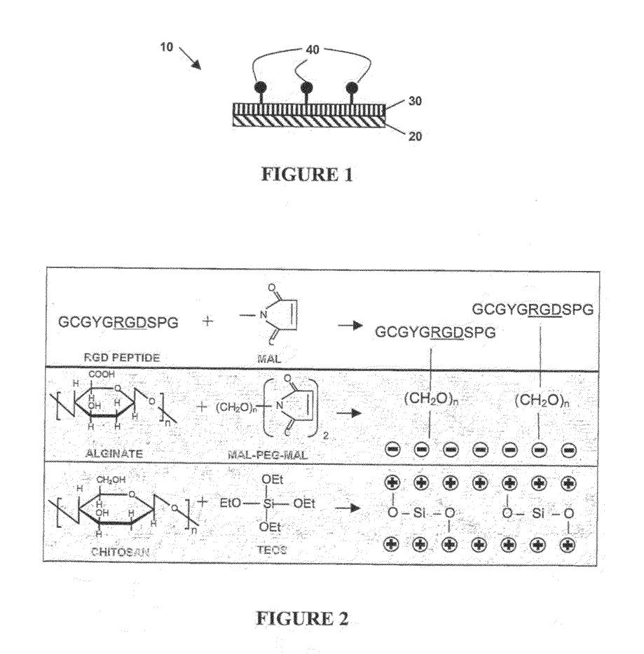

One embodiment of the present material is depicted in FIG. 1. The material 10 has a first polycationic layer 20. The polycationic layer 20 comprises any polycation. If the polycationic layer 20 is to come in contact with tissue, the polycation may be chosen to be biocompatible, non-cytotoxic and non-allergenic and so as to cause minimal irritation to tissue. A polycation or a cationic polymer, as used herein, refers to a polymer that possesses multiple positive charges at the pH of intended use, for example between pH 5 and 8 when intended for biological use. The polycation may be a biological polycation, such as a cationic polysaccharide, for example chitosan, or poly(arginine), poly(lysine), poly(ornithine), or another polycation such as a cationic organic polymer for example poly(ethyleneimine) or poly(allylamine).

Chitosan is a cationic polysaccharide derived from the crustacean exoskeleton. The use of chitosan as a biomaterial for drug delivery and tissue engineering applications has been widely investigated due to its biocompatibility and biodegradability.⁶

The polycation may optionally be cross-linked. Cross-linking of the polycationic layer 20 may result in reduced swellability of the resultant material, which may be desirable for certain applications. Furthermore, cross-linking of the polycationic layer may result in a stronger material.

Various methods of cross-linking chitosan and other polycations exist. The most common method in the art involves the use of a dialdehyde (e.g. glutaraldehyde) to cross-link a polymer via amine functionalities. However, it may be desirable not to introduce glutaraldehyde or other amine-reactive groups into the present material. Furthermore, amine groups may be contributing to the positive charge of the polycation, and thus any cross-linking reaction affecting these amino functionalities may be undesirable.

Therefore, the cationic polymer may be cross-linked using a cross-linker that does not react with amines or with carboxyl groups. For example, cross-linkers that link the polycation via hydroxyl or sulfhydryl groups, where such functional groups exist on the cationic polymer, may be used.

One example of such a cross-linking agent is hydrolysed tetraethyl orthosilicate (TEOS). Such silica cross-linking involves the condensation reaction between the hydroxyl groups (or residual ethoxy groups) of the hydrolyzed TEOS precursor and the hydroxyl groups of chitosan. Silica cross-linking may further act to improve the adhesion of the polycationic layer 20 to hydroxyl-rich surface of substrates, such as glass coverslips treated with ‘Piranha’ solution (i.e. a mixture of 30% H₂O₂ and 70% concentrated H₂SO₄).

The material 10 further comprises a polyanion-polyethylene glycol layer 30. This polyanion-polyethylene glycol layer 30 comprises a polyanion conjugated to polyethylene glycol.

The polyanion is any polyanion that is biocompatible, non-toxic, non-allergenic and that causes minimal irritation to tissue. The term polyanion or anionic polymer, as used herein, refers to a polymer that possesses multiple negative charges at the pH of intended use, for example between pH 5 and 8 when intended for biological use. The polyanion may be a biological polyanion, for example an anionic polysaccharide, for example heparin, alginic acid or hyaluronic acid, or another polyanion, for example an anionic organic polymer such as poly(acrylic acid), poly(methacrylic acid) or poly(acrylic-co-methacrylic acid).

The polyethylene glycol may be any polyethylene glycol, including a derivatized polyethylene glycol. For example, the polyethylene glycol may be derivatized with one or more maleimide functionalities, for example MAL-PEG-MAL. The polyethylene glycol may be of any average molecular size, and in certain embodiments is from about 400 to about 40,000 daltons in average molecular weight.

The polyethylene glycol moiety of the polyanion-polyethylene glycol layer 30 provides a non-fouling surface, which has reduced affinity for non-specific binding of proteins, thereby reducing the non-specific adherence of proteins and/or cells to the surface of the material. Without being bound by theory, the non-fouling properties of polyethylene glycol may result from a steric repulsion between the polyethylene glycol surface and proteins.

The polyanion is conjugated to the polyethylene glycol. The conjugation reaction may occur so as to form a block co-polymer, although the resulting conjugate may form a branched structure. The ratio of polyanion to polyethylene glycol in the polyanion-polyethylene glycol layer 30 may be varied, depending on the desired degree of negative charges or non-fouling surface. For example, the polanion:polyethylene glycol ratio may be from about 10:1 to about 1:10, or from about 5:1 to about 1:5 or from about 2:1 to about 1:2, or about 1:1.

The conjugation between the polyanion and the polyethylene glycol may be performed between non-carboxylic and non-amino functional groups. This reduces use of carboxylic acid groups that may be contributing to the negative charge of the polyanion in the conjugation reaction, and lessens use of reactive functional groups that may interact with biological molecules such as proteins, since the most common functional groups in proteins are carboxyl and amino groups. Since conjugation reactions involving carboxyl groups or amino groups typically require a cross-linker that reacts with similar groups on proteins, exclusion of such a cross-linker and use of non-carboxylic and non-amino functional groups on the polyanion and the polyethylene glycol should exclude cross-reaction with proteins, including cell-surface proteins.

Thus, other functional groups, such as for example hydroxyl groups or thiol groups may be included in either of the polyanion or the polyethylene glycol. For example, the polyanion can be modified to include a thiol group, such as by derivitization of the polyanion with cysteine. Particular examples of cysteine-modified polyanions that are suitable for inclusion in the present material are cysteine-heparin and cysteine-alginate, in which heparin or alginate has been reacted with cysteine. The cysteine substitution on the polyanion may be varied depending on the desired degree of inclusion of groups capable of reacting with the polyethylene glycol. For example, the cysteine substitution of the polyanion may be from about 100 to about 1000 μmol per gram of polyanion, or from about 200 to about 600 μmol per gram of polyanion.

To allow for the conjugation of the polyanion and the polyethylene glycol, a complementary functional group is included on the other of the polyanion and polyethylene glycol. For example, if the polyanion includes a thiol group, the polyethylene glycol is modified to contain a group that reacts with a free thiol group, such as a thiol group, a maleimide group, or a halogen derivative such as haloacetyl, benzyl halide that reacts through a resonance activation process with the neighboring benzene ring or alkyl halide that possesses the halogen β to a nitrogen or sulfur atom. In a particular example, polyethylene glycol modified with a maleimide functionality is used. In another particular embodiment, MAL-PEG-MAL is used.

The polyanion-polyethylene glycol layer 30 is adjacent to the polycation layer 20, and binds to the polycation layer 20 through electrostatic interactions between the negative charges of the polyanion moiety and the positive charges of the polycationic layer 20. In the present context, when one layer of the material is “adjacent to” another layer, the layers are immediately next to each other, and the layers may be covalently bound to each other, connected by electrostatic interactions, or merely physically touching each other.

The polyanion-polyethylene glycol layer 30 has a ligand 40 conjugated to the surface of the layer that is not adjacent to polycationic layer 20. The ligand 40 is any ligand that is specific for any binding molecule that is desired to be bound to the surface of the material. For example, the ligand 40 may be a ligand for a cell surface receptor found on a particular cell type, an enzyme, a substrate for a protein such as an enzyme or a receptor, including a peptide substrate such as a hormone, or the ligand 40 may be an antigen for binding a given antibody.

In one particular example, ligand 40 is a peptide containing the sequence RGD. Since its identification as a primary attachment cue by Pierschbacher and Ruoslahti,¹⁵ the RGD sequence has been widely applied in the biomaterials field.¹⁶⁻¹⁹ This sequence binds to the integrin receptor on a variety of cell types, including the kidney epithelial cells. In a particular example, the ligand 40 is a peptide comprising the sequence GCGYGRGDSPG [SEQ ID NO.: 1], or is a peptide consisting of the sequence GCGYGRGDSPG or consisting essentially of the sequence GCGYGRGDSPG. In the present context, it will be understood that what is meant by a peptide consisting essentially of a given sequence is a peptide that may have one, two, three or a few additional amino acids at either or both ends of the sequence, but that the additional amino acids do not materially affect the ability of the sequence to act as a ligand or recognition sequence. For example, a peptide consisting essentially of the sequence set out in SEQ ID NO.: 1 may have one, two, three or more additional amino acids at either end of the sequence defined above (or both ends), but such additional amino acids will not alter or influence the ability of the above sequence to act as an RGD peptide that binds to the integrin receptor.

The ligand 40 is conjugated to the polyanion-polyethylene glycol layer 30 by a reaction between non-carboxyl, non-amino groups on the ligand and the polyanion-polyethylene glycol layer 30. For example, if a peptide possessing a cysteine residue is used as the ligand 40, the free thiol group of the cysteine may be reacted with an appropriate functional group in the polyanion-polyethyleneglycol layer 30, such as a free thiol or a maleimide group that has not been consumed during the conjugation of the polyanion with the polyethylene glycol. In a particular embodiment, the ligand is conjugated to the polyethylene glycol to allow for good accessability of the ligand for binding.

Inclusion of a specific ligand to direct cell adhesion, conjugated to the polyanion-polyethylene glycol layer 30, for example to the non-fouling surface of the material, helps to minimise non-specific protein and cell adhesion, and ensures that the ligand is uniformly oriented at the surface of the material. Besides adhesion, this allows for immobilization of other biological functionalities on the surface of the material.

In the present strategy to produce modified membranes and coatings, conjugation involving amino and carboxyl groups can be deliberately excluded, including in the cross-linking of the polycationic layer and in the conjugation of ligands to the polyanion-polyethylene glycol layer. The chemical strategy employing the thiol/maleimide functionality is deemed advantageous as it does not require carbodiimide chemistry (e.g. EDC/N-hydroxy succinimide (NHS) coupling) for conjugation via the carboxyl group, which may disrupt the electrostatic interactions within the layers of the material. In addition, as the maleimidyl functionality is known to be specific towards thiol groups and not amino groups, the orientation of ligands can be better controlled by the introduction of cysteine residues in the protein or peptide molecules to be conjugated. Similarly, side-reactions such as protein cross-linking, can be avoided in this approach.

This approach presents several advantages. Proteins themselves typically contain numerous amino and carboxyl moieties, thus, the use of activators of amide formation such as EDC and NHS would pose a real danger of deactivating the protein function. In contrast, better selectivity can be achieved by using a functional group such as the maleimidyl functionality, which reacts exclusively with thiol groups. The relative stability of the maleimidyl group in water is also advantageous. Conventional conjugation reactions involving carbodiimides are negatively impacted by water hydrolysis.¹⁴

Furthermore, when designing the material, suitable polycations and polyanions can also be chosen to enhance or to reduce the interaction of cells with the material surface. Use of polyelectrolyte membranes is an approach that is quickly finding its way into biomaterial applications due to its convenience of application and ability to provide desirable surfaces. For example, the long-term stability of polyelectrolyte multilayers based on polyacrylamide and poly(acrylic-co-methacrylic acid) has been found to be exceptionally good, and protein adsorption onto these layers was relatively low.⁷ Electrostatic self-assembly employing combinations of poly(ethyleneimine), gelatin and chitosan has been used to promote osteoblast growth on poly(DL-lactide)⁸ and titanium⁹ substrates, while a multilayer composed of collagen and hyaluronic acid has been used to grow chondrosarcoma cells.¹⁰ Thus, based on these results, it is possible to direct the nature of the various layers of the present material, and thus direct the nature of the material as a whole.

In alternate embodiments, additional polyelectrolyte layers may be included, with each subsequent layer having the opposite charge to an adjacent layer such that the multiple layers are held together through electrostatic interactions. Additional negatively charged layers may comprise polyanions, without the need to include polyethylene glycol in the layer. Additional layers are thus added to the above-described embodiment adjacent to polycationic layer 20, on the opposite side as polyanion-polyethylene glycol layer 30, so as not to interfere or interrupt the surface with conjugated ligand 40.

Optionally, the material may include a therapeutic agent incorporated into one or more of the polyelectrolyte layers. The therapeutic agent may be any agent that is to be delivered with the material. For example, the therapeutic agent may be a protein, a peptide, an enzyme, a growth factor, a hormone, a nucleic acid molecule, a small molecule, a drug, an antibiotic, an anti-inflammatory agent, an anti-clotting agent or a chemotherapeutic agent.

The above-described material 10 may be formed as follows. First, the polycationic layer 20 is formed. A polycation solution is prepared and is formed into a desired shape, for example by pouring into a cast or mold. The solution is allowed to dry, leaving the dry polycation:

If cross-linking of the polycation is desired, a solution containing the appropriate cross-linker and polycation is cast into the desired shape. The amount and concentration of cross-linker used can be varied, depending on the degree of cross-linking desired, as will be appreciated by a skilled person. For example, the cross-linker to monomer ratio, where the monomer is the building block of the polycation, may be from about 1:1000 to about 1:2, from about 1:100 to about 1:2, from about 1:10 to about 1:2. Alternatively, the polycationic layer can first be formed prior to subjecting it to the crosslinking treatment.

Once the desired form or shape of the polycationic layer 20 has been obtained and the polycation has been crosslinked, the polycationic layer 20 may be washed to remove excess crosslinker and solvent, for example with water or with a suitable non-reactive buffer that will not interfere with layering of subsequent layers of the material.

A polyanion-polyethylene glycol conjugate may be prepared by reacting a polyanion having suitable functional groups available for conjugation, for example a free sulfhydryl group, together with polyethylene glycol, the polyethylene glycol having a functional group that reacts with the available functional group in the polyanion. The two components may be reacted by mixing the two together in solution under conditions that allow for the reaction between the functional groups. The ratio of the two components may be varied in order to vary the amount of polyanion or polyethylene glycol in the resultant conjugate. For example, the polyanion to polyethylene glycol ratio may be from about 10 to 1 to about 1 to 10.

A solution containing the conjugate is then applied to the dry polycationic layer 20, and the solution is allowed to dry, leaving the polycationic layer 20 with the overlayer of the polyanion-polyethylene glycol layer 30. This bilayer may be rinsed to remove any excess conjugate. Alternatively, the conjugate solution may be applied to the dry polycationic layer 20 for a fixed time period, without drying, and the bilayer subsequently rinsed to remove excess conjugate.

Once the above bilayer is formed, a ligand 40 is conjugated to the polyanion-polyethylene glycol layer 30. As above, the ligand 40 is applied under suitable reaction conditions for a time sufficient to allow the conjugation of the ligand 40 to the polyanion-polyethylene glycol layer 30. The concentration of ligand 40 is adjusted to allow for the appropriate degree of conjugation. A skilled person can readily determine, using routine laboratory methods, the degree of conjugation of ligand 40 on the polyanion-polyethylene glycol layer 30. If desired, the resultant material may be rinsed to remove any excess unconjugated ligand.

The present material 10 may be formed as a membrane to use as a two-dimensional surface for cell growth and support. Alternatively, the present material 10 may be formed onto a device or implant to provide such a device or implant with a surface that is suitable for attachment of a specific cell type, for example liver or kidney epithelial cells. When coating a device or implant, the various layers of the material 10 may be formed directly on the device or implant. Alternatively, the material 10 may be preformed and then shaped and applied to the surface of a device or implant.

When applying the material 10 to a particular surface, the material 10 can be designed so that the surface of the material 10 that is to contact the surface of the solid support or device has charges or groups that will bind to complementary charges or groups on the solid support. For example, plasma treated glass surfaces would be rich with hydroxyl and carboxyl groups which, being anionic in nature, could interact with positive charges of a polycationic layer 20 located on the opposite side of the material 10 from the polyanion-polyethylene glycol layer 30. Alternatively, if the solid support surface has positively charged groups on the surface, the material 10 may be designed with sufficient number of layers such that the surface of the material 10 that is opposed to the ligand-conjugated surface is a polyanionic layer, to improve adherence of the material 10 onto the solid support surface.

Also presently contemplated are articles of manufacture incorporating the above-described material, for example a membrane comprising the present material, or an implantable medical device comprising the material. The membrane or implantable medical device is coated with the present material on any surface that is intended to come into contact with cells or body fluid or tissue, so as to select and/or direct the adhesion of biomolecules and cells to the membrane or implantable device.

Also presently contemplated are methods of adhering a biological molecule or cell to a surface using the described material. The surface to which the biological material or cell is to be adhered is coated with the material, with the ligand on the outer-most surface of the material, making the ligand available to be bound by the target biological molecule or cell. As will be appreciated, the ligand is selected such that it will selectively bind the desired biological molecule or cell type, while minimising or reducing non-specific binding of other biological molecules or cells.

The invention is further exemplified by the following non-limiting examples.

### EXAMPLES

### Example 1

### Materials and Methods

Polyelectrolyte Complex Membrane:

Casting of Membranes: Membranes were cast from a solution of 1% w/v chitosan in 2% w/v acetic acid (HOAc) in polypropylene molds, and allowed to coagulate and dry in the fume hood for 1-2 days. Hydrolyzed TEOS (Fluka) was prepared by mixing 1 part TEOS in 9 parts 0.15 M HOAc by volume, and vortexing for 1 h, or until only one phase was present. Typically, hydrolyzed TEOS was incorporated into the chitosan solution at a volume ratio of 1:3. A biopsy punch was used to cut the membrane into circular disks (6 mm-diameter) for the swelling studies. For the cell adhesion studies, the membranes were clamped within Minutissue™ rings of 7 mm I.D., and the chemical reactions were performed on one surface. Swelling studies were performed by immersing membranes in deionized water, and measuring the diameter at regular time intervals until no more swelling occurred, which was typically within 6 h.

Polyanion-PEG Conjugate: Cysteine-alginate was synthesized as reported by Bernkop-Schnürch and co-workers.⁵ Briefly, a 1% w/v solution of low molecular weight alginic acid (Sigma) was prepared in deionized water. 1-ethyl-3-(3-dimethylaminopropyl)carbodiimide (EDC, Merck) was added to the solution at a final concentration of 50 mM, and allowed to react for 45 min. An equal volume of 0.5% w/v solution of 1-cysteine monohydrate hydrochloride (Merck) was then added dropwise to the mixture under stirring, and the pH was adjusted to 4.0. The resulting mixture was stirred for 2 h at room temperature before the pH was raised to 6.0 and reacted for one more hour. Cysteine-alginate was purified by dialyzing (Spectrum Laboratories, MWCO 3500) the mixture against 1 mM hydrochloric acid (HCl, Merck) for 1 h at 4° C. This was followed by dialyzing twice against 1 mM HCl containing 1% w/v sodium chloride (NaCl, Merck) for 1 h each at 4° C. and, finally, dialyzing overnight against 1 mM HCl at 4° C. The purified product was isolated by lyophilization (VirTis BenchTop 4K Freeze Dryer).

To determine the degree of cysteine substitution, 1 mg of the lyophilized product was dissolved in 1 ml of deionized water, and the pH was adjusted to between 2 and 3. 200 μl of 1% w/v starch (Merck) solution was added to the solution, and the mixture was titrated against a 1.00 mM aqueous iodine (Merck) solution till a permanent pale blue color was observed.

The preparation of cysteine-heparin followed a similar procedure as that of cysteine-alginate, except that heparin (Sigma) was used instead of alginic acid. The degrees of cysteine substitution in cysteine-alginate and cysteine-heparin were determined by iodometric titration to be 254 and 579 μmol/g of polymer, respectively.

To form the polyanion-PEG conjugate, 5 mg of cysteine-alginate or cysteine-heparin were reacted with 5.5 mg of MAL-PEG-MAL (Nektar) in 0.5 mL of deionized water by mixing equivolume solutions of the two reactants.

Preparation of Membrane: 50 μL of the polyanion-PEG conjugate were applied to the silica-cross-linked polycationic membrane clamped in a Minutissue ring at room temperature. After 1 h, the solution was evaporated, and the membrane was rinsed thrice with deionized water to remove the excess polyanion-PEG conjugate. The RGD peptide, GCGYGRGDSPG (Mimotopes) [SEQ ID NO: 1], was conjugated by applying 50 μL of a 1 mg/mL peptide solution uniformly to the surface of the membrane. After 1 h of reaction, the membrane was rinsed thrice with deionized water.

Polyelectrolyte Complex Coatings: Hydroxyl groups were generated on glass surfaces using either of the following two methods. Glass coverslips were immersed in a ‘Piranha’ solution (i.e. a mixture of 30% H₂O₂ and 70% concentrated H₂SO₄) for 1 h at 100° C., rinsed with deionized water, and dried under an air stream. Alternatively, glass coverslips were cleaned in a RBS 35® detergent solution at 50° C. for 30 min. Each glass coverslip (2.2 cm×2.2 cm) was subsequently coated with chitosan by uniformly applying 100 μL of a 3:1 chitosan:hydrolyzed TEOS solution (0.5% w/v chitosan solution in 2% w/v HOAc, 1:9 TEOS:0.15 M acetic acid) on its surface. Following this, the polyanion-PEG conjugate and RGD peptide were applied as described in the membrane preparation.

Cell Adhesion Studies:

Cell Culture: Primary human cortical renal cells were obtained from Cambrex (Walkersville, Md., USA). The proximal tubule cells were cultured in REGM (Cambrex, Walkersville, Md., USA) under 5% CO₂ at 37° C. HepG2 cells were obtained from ATCC, and cultured in full DMEM media (Sigma, St Louis, Mo., USA). Membranes and coverslips were sterilized by immersion in 70% ethanol for at least 30 min, followed by exposure to ultraviolet light for 30 min. Cells were seeded at a density of 7.5×10⁴ cells per 24-well plate.

Cell Staining and Immunohistochemistry: The adhered cells were fixed in ice-cold ethanol for 10 min. After several rinses with phosphate-buffered saline (PBS), the samples were incubated with blocking solution containing PBS, 10% fetal calf serum (FCS) and 1% bovine serum albumin (BSA) for 30 min. The AQP1 primary antibody (Santa Cruz Biotechnologies, Santa Cruz, Calif., USA) was diluted at a ratio of 1:100 and incubated for 2 h. After several rinses, the specimens were incubated for 45 min with donkey-anti-rabbit-IgG-FITC-conjugated secondary antibody (Jackson Immunoresearch Laboratories, West Grove, Pa., USA), which had been diluted 200-fold in PBS containing 1% BSA. Nuclear staining was done with 4′,6-diamidino-2-phenylindole (DAPI, Sigma-Aldrich, Singapore). The specimens were then analyzed using an IX71 Olympus microscope (Tokyo, Japan).

Results:

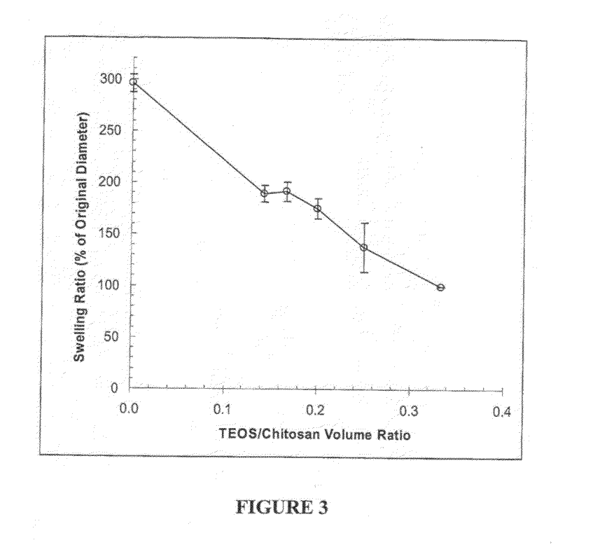

Chitosan-TEOS: Significant membrane swelling was observed in the absence of hydrolyzed TEOS. Swelling was reduced when hydrolyzed TEOS was introduced (see FIG. 3). A hydrolyzed TEOS:chitosan volume ratio of 1:3 was used in our synthesis to avoid membrane swelling. This study illustrated that cross-linking with silica physically strengthened the chitosan-based membrane.

Polyanion-PEG Conjugate: The intermediate layer of the membrane was comprised of a polyanion-PEG conjugate. As the anionic carboxyl groups of the polyanion were required for the electrostatic interaction with chitosan, we have used thiol addition to form the polyanion-PEG conjugate. The cysteinylated derivatives of both alginate and heparin were synthesized as a prerequisite. They were reacted with MAL-PEG-MAL, a PEG that is bifunctional with respect to the thiol-reactive maleimidyl end group. The polyanion-PEG conjugate was then layered onto the chitosan-based membrane or coating, followed by rinsing to remove the excess, unreacted conjugate.

Conjugation of Ligand: Thiol chemistry was chosen as the basis of reaction between the intermediate polyanion-PEG conjugate and the ligand to be presented. The maleimidyl groups present in the conjugate were reacted with the thiol-containing cysteine residues in the RGD peptide sequence. This chemical strategy does not require EDC/N-hydroxy succinimide (NHS) chemistry for conjugation via the carboxyl group, which may disrupt the electrostatic interactions within the membrane or coating. In addition, as the maleimidyl functionality is known to be specific towards thiol groups and not amino groups, the orientation of ligands can be better controlled by the introduction of cysteine residues in the protein or peptide molecules to be conjugated. Similarly, side-reactions such as protein cross-linking, can be avoided in this approach.

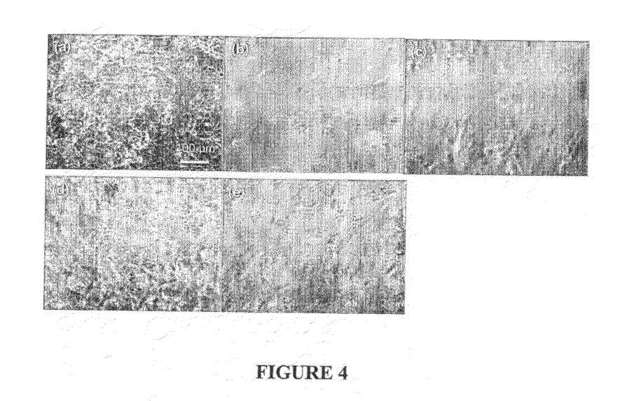

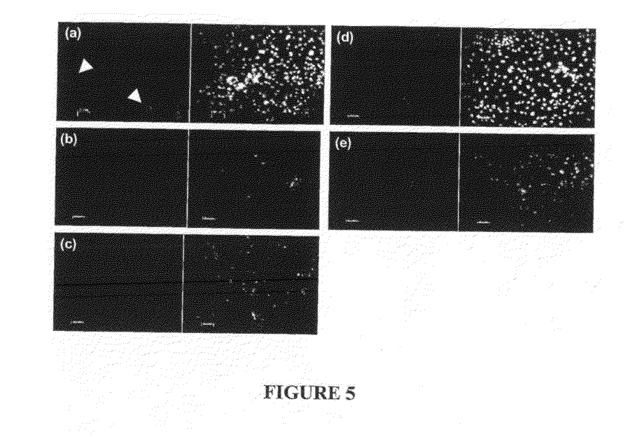

Cell Culture on RGD Modified Membrane or Coating: After 2 weeks of culture, the density of primary human cortical renal cells grown on the various membranes showed the following trend: chitosan-alginate-PEG-RGD, chitosan-heparin-PEG-RGD>chitosan-heparin-PEG, chitosan>chitosan-alginate-PEG (FIG. 4). The renal cells were fully confluent on surfaces that were RGD-modified, regardless of whether alginate-PEG-MAL or heparin-PEG-MAL had been used as the intermediate layer. The effect of the polyanionic intermediate layer on cell adhesion was illustrated by comparing alginate-PEG-MAL and heparin-PEG-MAL surfaces to the unmodified chitosan surface. Much poorer cell adhesion was observed on alginate-PEG-MAL (FIG. 4(b)) compared to the unmodified chitosan (FIG. 4(c)), confirming the non-fouling properties of the former. In contrast, cell adhesion on heparin-PEG-MAL (FIG. 4(e)) appeared to be comparable to that on the chitosan surface. This might be due to the fact that heparan sulfate proteoglycans are a major component of the kidney ECM, and that heparin provides binding sites for both cell surface receptors and growth factors that positively influence cell adhesion^{20, 21}. Labelling of the water channel protein, AQP1, characteristic of the proximal renal tubule cell phenotype, confirmed the presence of AQP1 expressing cells on the membranes (see FIG. 5). The trend in AQP1 expression correlated well with the cell density, as indicated by the nuclear stain, DAPI.

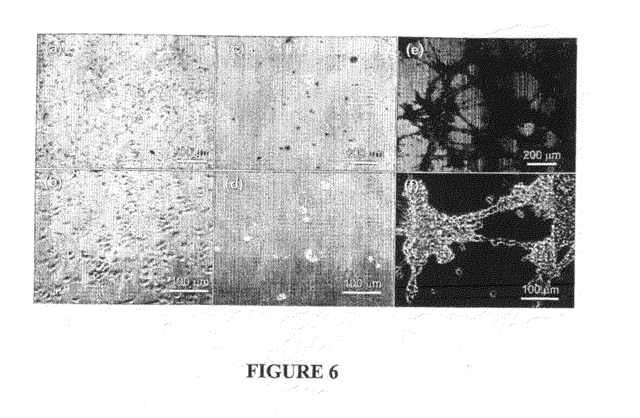

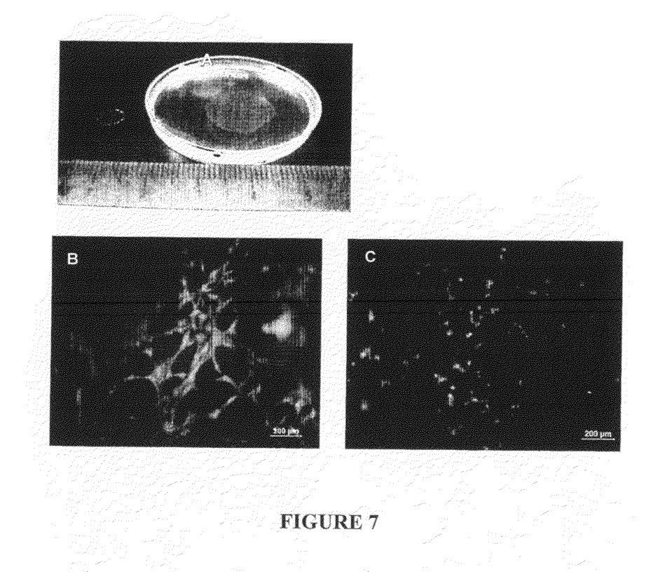

The effect of the various modified coatings on the growth of HepG2 is shown in FIG. 6. In this case, both the degree and the cell adhesion pattern were affected by the availability of the RGD ligand. While cells were attached and distributed evenly in the case of the uncoated glass surface (FIGS. 6(a) and (d)), they were hardly attached to the alginate-PEG-MAL surface (FIGS. 6(c) and (d)). In contrast, cells on the RGD-modified surface attached and proliferated in the form of islands, interconnected by a series of bridges (FIG. 6(e)). Within each island, cells were observed to aggregate into a tight formation (FIG. 6(f)), and each bridge was constituted of cords of cells. This phenomenon might be attributed to the presence of RGD, which transduced its signals on a basically non-adhesive surface. As cell spreading was restricted, cell proliferation must take place with minimal surface contact, causing the cells to aggregate. A second possibility might be the switching on of a signal that directed cell aggregation.²² In either case, RGD ligand was effectively presented to the cells to mediate their adhesion onto an otherwise non-fouling, non-adhesive surface.

### Example 2

### Cell-Adhesive Polyelectrolyte Membrane and Coating

A swellable form of the above-described membrane was cast from a “water soluble” chitosan (pH 4.8, viscosity=28 mPa·s, Degree of deacetylation=88%; NOF Corporation). As swelling was desired in this case, TEOS was not employed as a crosslinker. The membrane exhibited approximately 6-9 fold instant swelling upon immersion in deionized water (FIG. 7A). The same chemical conjugation strategy as above was used to immobilize RGD on the swellable membrane. Phosphate buffered saline was used in place of water for the rinsing steps in order to reduce further swelling and to maintain membrane integrity.

Human mesenchymal stem cells (hMSC) seeded onto the ROD-modified swellable membrane exhibited good adhesion and spreading of cells (FIG. 7B), as compared to cells seeded onto the alginate-PEG control membrane (no RGD modification) (FIG. 7C).

As can be understood by one skilled in the art, many modifications to the exemplary embodiments described herein are possible. The invention, rather, is intended to encompass all such modification within its scope, as defined by the claims.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### REFERENCES

1. G. Rh. Owen, J. Jackson, B. Chehroudi, H. Burt, D. M. Brunette. A PLGA membrane controlling cell behaviour for promoting tissue regeneration. Biomaterials 26 (2005) 7447-7456.
2. F. Stavropoulos, C. Dahlin, J. D. Ruskin, C Johansson. A comparative study of barrier membranes as graft protectors in the treatment of localized bone defects. Clin. Oral Impl. Res. 15 (2004) 435-442.
3. J. L. Ajuria, P. L. Kimmel. Choice of hemodialysis membrane, in Principles and Practice of Dialysis, 3rd Ed., W. L. Henrich, Ed., Lippincott Williams and Wilkins, Hong Kong, 2004.
4. L. P. K. Ang, Z. Y. Cheng, R. W. Beuerman, S. H. Teoh, X Zhu, D. T. H. Tan. The development of a serum-free derived bioengineered conjunctival epithelial equivalent using an ultrathin poly(ε-caprolactone) membrane substrate. Invest. Opthalmol. Vis. Sci. 47 (2006) 105-112.
5. A. Bernkop-Schnürch, C. E. Kast, M. F. Richter. Improvement in the mucoadhesive properties of alginate by the covalent attachment of cysteine. J. Control. Release 71 (2001) 277-285.
6. E. Khor, L. Y. Lim. Implantable applications of chitin and chitosan. Biomaterials 24 (2003) 2339-2349.
7. S. Y. Yang, J. D. Mendelsohn, M. F. Rubner. New class of ultrathin, highly cell-adhesion-resistant polyelectrolyte multilayers with micropatterning capabilities. Biomacromolecules 4 (2003) 987-994.
8. H. Zhu, J. Ji, M. A. Barbosa, J. Shen. Protein electrostatic self-assembly on poly(DL-lactide) scaffold to promote osteoblast growth. J. Biomed. Mater. Res. B: Appl. Biomater. 71B (2004) 159-165.
9: K. Cai, A. Rechtenbach, J. Hao, J. Bossert, K. D. Jandu Polysaccharide-protein surface modification of titanium via a layer-by-layer technique: Characterization and cell behaviour aspects. Biomaterials 26 (2005) 5960-5971.
10. J. Zhang, B. Senger, D. Vautier, C. Picart, P. Schaaf, J.-C. Voegel, P. Lavalle. Natural polyelectrolyte films based on layer-by-layer deposition of collagen and hyaluronic acid. Biomaterials 26 (2005) 3353-3361.
11. M. Mrksich. What can surface chemistry do for biology. Curr. Opin. Chem. Biol. 6 (2002) 794-797.
12. J. Groll, J. Fiedler, E. Engelhard, T. Ameringer, S. Tugulu, H.-A. Klok, R. E. Brenner, M. Moeller. A novel star PEG-derived surface coating for specific cell adhesion. J. Biomed. Mater. Res., 74(A) (2005) 607-617.
13. W.-C. Lin, T.-Y. Liu, M.-C. Yang. Hemocompatibility of polyacrylonitrile dialysis membrane immobilized with chitosan and heparin conjugate. Biomaterials 25 (2004) 1947-1957.
14. K. Yang, A. Basu, M. Wang, R. Chintala, M.-C. Hsieh, S. Liu, J. Hua, Z. Zhang, J. Thou, M. Li, H. Phyu, G. Petti, M. Mendez, H. Janjua, P. Peng, C. Longley, V. Borowski, M. Mehlig, D. Filpula. Tailoring structure-function and pharmacokinetic properties of single-chain Fv proteins by site-specific PEGylation. Protein Engineering, 16 (2003) 761-770.
15. M. Pierschbacher, E. Ruoslahti. Cell attachment activity of fibronectin can be duplicated by small synthetic fragments of the molecule. Nature 309 (1984) 30-33.
16. J. A. Rowley, G. Madlambayan, D. J. Mooney. Alginate hydrogels as synthetic extracellular matrix material. Biomaterials 20 (1999) 45-53.
17. S. Woerly, E. Pinet, L. de Robertis, D. van Diep; M. Bousmina. Spinal cord repair with PHPMA hydrogel containing RGD peptides (NeuroGel). Biomaterials 22 (2001) 1095-1101.
18. T. W. Chung, Y. F. Lu, S. S. Wang, Y. S. Lin, S. H. Chu. Growth of human endothelial cells on photochemically grafted Gly-Arg-Gly-Asp (GRGD) chitosans. Biomaterials 23 (2002) 4803-4809.
19. S. VandeVondele, J. Voros, J. A. Hubbell. RGD-grafted poly-L-lysine-graft-(polyethylene glycol) copolymers block non-specific protein adsorption while promoting cell adhesion. Biotechnol. Bioeng. 82 (2003) 784-790.
20. A. J. Groffen, M. A. Ruegg, H. Dijkman, T. J. van de Velden, C. A. Buskens. Agrin is a major heparan sulfate proteoglycan in the human glomerular basement membrane. J. Histochem. Cytochem. 46 (1998) 19-27.
21. A. K. Powell, D. G. Fernig, J. E. Turnbull. Fibroblast growth factor receptors 1 and 2 interact differently with heparin/heparan sulphate. J. Biol. Chem. 277 (2002) 28554-28563.
22. F. Monier-Gavelle, J.-L. Duband. Cross talk between adhesion molecules: Control of N-cadherin activity by intracellular signals elicited by 131 and (33 integrins in migrating neural cells. J. Cell Biol. 137 (1997) 1663-1681.

What is claimed is:

1. A support material comprising:
a polyelectrolyte layer comprising a first polyelectrolyte;
a polyelectrolyte-polyethylene glycol layer adjacent to said polyelectrolyte layer, said polyelectrolyte-polyethylene glycol layer comprising a second polyelectrolyte being of opposite charge to said first polyelectrolyte, said second polyelectrolyte conjugated to polyethylene glycol by a first functional group on said second polyelectrolyte and a second functional group on said polyethylene glycol; and
a ligand conjugated to said polyelectrolyte-polyethylene glycol layer by a third functional group on said ligand and a fourth functional group on said polyelectrolyte-polyethylene glycol layer,
wherein neither of said third or fourth functional groups are a carboxyl group or an amino group.

2. The support material of claim 1 wherein said first polyelectrolyte is a polycation and said second polyelectrolyte is a polyanion.

3. The support material of claim 1 or claim 2 wherein neither of said first or second functional groups are a carboxyl group or an amino group.

4. The support material of any one of claims 1 to 3 wherein the first polyelectrolyte comprises a biological polycation, a cationic polysaccharide, a polypeptide or a cationic organic polymer.

5. The support material of any one of claims 1 to 4 wherein the first polyelectrolyte comprises chitosan, poly(arginine), poly(lysine), poly(ornithine), poly(ethyleneimine) or poly(allylamine).

6. The support material of any one of claims 1 to 5 wherein the first polyelectrolyte comprises chitosan.

7. The support material of claim 6 wherein the chitosan is cross-linked

8. The support material of claim 7 wherein the chitosan is cross-linked with hydrolysed tetraethyl orthosilicate.

9. The support material of any one of claims 1 to 8 wherein the second polyelectrolyte comprises a biological polyanion, an anionic polysaccharide or an anionic organic polymer.

10. The support material of any one of claims 1 to 9 wherein the second polyelectrolyte comprises heparin, alginic acid, poly(acrylic acid), poly(methacrylic acid), poly(acrylic-co-methacrylic acid) or hyaluronic acid.

11. The support material of any one of claims 1 to 10 wherein the second polyelectrolyte comprises heparin or alginate.

12. The support material of claim 11 wherein the heparin or alginate is derivatized with cysteine.

13. The support material of claim 12 wherein the polyethylene glycol is modified with maleimide.

14. The support material of claim 13 wherein the polyethylene glycol is MAL-PEG-MAL.

15. The support material of any one of claims 1 to 14 where in the ligand is a ligand for a cell surface receptor, an enzyme, a substrate for an enzyme, a hormone, or an antigen.

16. The support material of any one of claims 1 to 14 wherein the ligand is a peptide comprising the sequence RGD.

17. The support material of claim 16 wherein the ligand is a peptide comprising the sequence of SEQ ID NO: 1.

18. The support material of claim 17 wherein the ligand is a peptide consisting of the sequence of SEQ ID NO: 1.

19. The support material of any one of claims 1 to 18 wherein either of said first functional group and said second functional group is a thiol group and the other of said first functional group and said second functional group is a maleimide group.

20. The support material of any one of claims 1 to 19 wherein said third functional group is the same type of functional group as said first functional group or said second functional group.

21. The support material of any one of claims 1 to 20 wherein said fourth functional group is the same type of functional group as said first functional group or said second functional group.

22. The support material of any one of claims 1 to 21 wherein said third functional group is a thiol group and said fourth functional group is a maleimide group.

23. The support material of any one of claims 1 to 22 wherein said polyelectrolyte layer is a first polyelectrolyte layer, further comprising a second polyelectrolyte layer of opposite charge to said first polyelectrolyte layer, said second polyelectrolyte layer adjacent to said first polyelectrolyte layer and on the opposite side of said polyelectrolyte layer from said polyelectrolyte-polyethylene glycol layer.

24. The support material of claim 22 or claim 23 further comprising a therapeutic agent.

25. The support material of claim 24 wherein the therapeutic agent is a protein, a peptide, an enzyme, a growth factor, a hormone, a nucleic acid molecule, a small molecule, a drug, an antibiotic, an anti-inflammatory agent, an anti-clotting agent or a chemotherapeutic agent.

26. A method of forming the support material of any one of claims 1 to 25 comprising:
providing a first layer comprising a first polyelectrolyte;
applying a polyelectrolyte-polyethylene glycol conjugate to said first layer to form a second layer adjacent to said first layer, where in said polyelectrolyte-polyethylene glycol conjugate is formed by reacting a first functional group on a second polyelectrolyte and a second functional group on a polyethylene glycol, said second polyelectrolyte being of opposite charge to said first polyelectrolyte; and
conjugating a ligand to said second layer by reacting a third functional group on said ligand with a fourth functional group on said second layer;
wherein neither of said third or fourth functional groups are a carboxyl group or an amino group.

27. The method of claim 22 wherein neither of first or second functional groups are a carboxyl group or an amino group.


SEQUENCE LISTING (1 sequence)

SEQ ID NO: 1 - protein, 11 aa, Artificial Sequence
  synthetic RGD peptide sequence
GCGYGRGDSPG